(19) United States
(12) Patent Application Publication
(10) Pub. No.: US 2017/0042823 A1
(43) Pub. Date: Feb. 16, 2017

(54) CO-ENCAPSULATION OF ANTIMICROBIALS AND ADJUVANTS IN NANOCARRIERS

(71) Applicant: THE TRUSTEES OF PRINCETON UNIVERSITY, Princeton, NJ (US)

(72) Inventors: Robert K. PRUD'HOMME, Lawrenceville, NJ (US); Hoang D. Lu, Woodside, NY (US)

(21) Appl. No.: 15/237,219

(22) Filed: Aug. 15, 2016

Related U.S. Application Data
(60) Provisional application No. 62/205,306, filed on Aug. 14, 2015.

(51) Int. Cl. A61K 9/50; A61K 33/38; A61K 31/05; A61K 9/51; A61K 31/20; A61K 31/496; A61K 38/12; A61K 9/10

(52) U.S. Cl. CPC A61K 9/5031; A61K 38/12; A61K 33/38; A61K 9/10; A61K 9/5123; A61K 31/20; A61K 31/496; A61K 31/05

(57) ABSTRACT

Microbial infections have become increasingly difficult to treat due to the emergence of drug resistant microbes. Adjunctive therapies can be used to better treat resistant microbes, where multiple drugs are concurrently used to overcome resistant mechanisms and to synergistically treat infections. The practice of adjunctive therapies is limited by the ability to precisely control the pharmacokinetic profiles of the multiple actives. Composite particle-based approaches to enable and enhance adjunctive antimicrobial infections by simultaneous encapsulation and delivery of all components are described herein.

### RELATED APPLICATION DATA

The present application claims priority pursuant to 35 U.S.C. §119(e)(1) to U.S. Provisional Patent Application Ser. No. 62/205,306 filed Aug. 14, 2015 which is incorporated herein by reference in its entirety.

### FIELD

The present invention relates to compositions and methods for improved adjunctive therapy and improved co-administration of active agents. More particularly, the invention addresses particulate constructs that co-encapsulate a primary antimicrobial active with at least one adjuvant to treat microbial infections.

### BACKGROUND

Adjunctive antimicrobial therapies against microbial infections can provide improved therapeutic outcomes over traditional courses of treatment. In adjunctive antimicrobial therapies, one antimicrobial active is concurrently administered with one or more additional active agent called an adjuvant. Adjuvants enhance the activity of the antimicrobial agent. These antimicrobial and adjuvant agents can have the same or different mechanisms of action to treat the disease by providing additive or synergistic drug effects. Adjunctive therapies are increasingly important for the treatment of antimicrobial resistant organisms. In the context of treating antimicrobial resistant infections, adjuvants can overcome or overwhelm resistance or tolerance mechanisms to allow antimicrobial agents to be effective towards otherwise resistant microbes.

Successful adjuvant therapy requires successfully delivering all the necessary components to the target sites at the correct concentrations and durations. However, administration of adjuvant therapies can be challenging since the necessary concentrations and durations of delivery, stabilities, pharmacokinetic properties, and pharmacodynamics properties of each component can vary extensively. The difficulty of successfully co-delivering all the agents required for adjuvant therapies can make otherwise promising methods of treatment not viable. Excessively rapid clearance or excessively slow release of even one component can result in therapy failure. The use of excessively high concentrations of one agent to meet the required concentrations for therapy can result in toxic side-effects. In addition, regimens for adjuvant therapies in which in which the optimal dosing schedules of agents with differing toxicities are determined using extensive post-marketing clinical trials. A technology that provides the ability to simultaneously and locally co-deliver all the components required for adjuvant microbial therapies at controlled rates can greatly enhance the efficacy of treatments.

### SUMMARY

In one aspect, compositions are described for the co-delivery of antimicrobial agent and adjuvant from a singular composite particle construction. For example, a composite particle described herein comprises a core component and a surface component, the core component comprising at least one antimicrobial agent and at least one antimicrobial adjuvant. As used herein, an antimicrobial adjuvant is a species that enhances the efficacy of the antimicrobial agent. For example, in some embodiments, antimicrobial adjuvants alone do not effectively kill the microbe treated and/or do not effectively inhibit the growth of the microbe treated at administered concentrations. Moreover, an antimicrobial adjuvant can be an agent that when co-encapsulated with an antimicrobial agent results in a composite particle having an antimicrobial combination index (CI) less than 1 or less than 0.5. In some embodiments, an antimicrobial adjuvant can sensitize pathogens to concurrently delivered antimicrobial agent(s), thereby enhancing antimicrobial agent efficacy while minimizing required dose of the antimicrobial agent. Antimicrobial adjuvant, in some embodiments, can also function by decreasing microbial virulence, by enhancing pharmacokinetic properties of the co-antimicrobial agent, by increasing uptake of the antimicrobial, decreasing efflux of the antimicrobial and/or by enhancing the native immune system. In some embodiments, the surface component of the composite particle comprises one or more amphiphilic stabilizers encapsulating the core component.

In another aspect, methods of treating microbial infections are described herein. A method of treating a microbial infection, in some embodiments, comprises administering to a patient or animal in need thereof a therapeutically effective amount of a composition comprising composite particles, the composite particles including a core component and a surface component, the core component comprising at least one antimicrobial agent and at least one antimicrobial adjuvant.

These and other embodiments are further described in the following detailed description.

### BRIEF DESCRIPTION OF THE DRAWINGS

FIG. 1 illustrates dynamic light scattering particle size distribution of composite nanoparticles comprising cis-2-decenioc acid and intraconazole according to some embodiments.

FIG. 2 illustrates dynamic light scattering particle size distribution of composite nanoparticles comprising farnesol and intraconazole according to some embodiments.

FIG. 3 illustrates dynamic light scattering particle size distribution of composite nanoparticles comprising silver colloid and intraconazole according to some embodiments.

FIG. 4 illustrates dynamic light scattering particle size distribution of composite nanoparticles comprising cis-2-decenoic acid and totarol according to some embodiments.

FIG. 5 illustrates dynamic light scattering particle size distribution of composite nanoparticles comprising polymyxin and totarol according to some embodiments.

FIG. 6 illustrates dynamic light scattering particle size distribution of composite nanoparticles comprising cis-2-decenoic acid and rifampicin prodrugs according to some embodiments.

FIG. 7 illustrates dynamic light scattering particle size distribution of composite nanoparticles comprising silver colloids and rifampicin prodrugs according to some embodiments.

FIG. 8 illustrates dynamic light scattering particle size distribution of composite nanoparticles comprising polymyxin and rifampicin prodrugs according to some embodiments.

FIG. 9 illustrates dynamic light scattering particle size distribution of the composite nanoparticles comprising co-encapsulation of silver colloids with totarol taken at time zero, three hours, 1 day, 2 day and 3 days of the storage according to some embodiments.

FIG. 10 is a transmission electron microscopy (TEM) image of oleic acid coated silver colloids employed in composite nanoparticles according to some embodiments.

FIG. 11 provides TEM images of composite nanoparticles co-encapsulating oleic acid coated silver colloids and totarol according to some embodiments.

FIG. 12 illustrates absorbance spectra of composite nanoparticles that co-encapsulate oleic acid coated silver colloids with totarol and the absorbance spectra of the flow-through fraction of nanoparticles that co-encapsulate oleic acid coated silver colloids with totarol when passed through a 10 kDa ultrafiltration filter according to some embodiments.

FIG. 13 illustrates the normalized absorbance peak maxima in the visible wavelength region over time when composite nanoparticles are diluted into phosphate buffered saline according to some embodiments.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In one aspect, compositions are described for the co-delivery of antimicrobial agent and adjuvant from a singular composite particle construction. For example, a composite particle described herein comprises a core component and a surface component, the core component comprising at least one antimicrobial agent and at least one antimicrobial adjuvant. Moreover, the surface component can comprise one or more amphiphilic copolymers encapsulating the core component. Encapsulation of the core component by the surface component can provide a core-shell architecture. In some embodiments, a composite particle further comprises one or more targeting moieties, one or more visualization or contrast agents and/or one or more components that influence the delivery or activity of the antimicrobial agent and/or antimicrobial adjuvant. Composite nanoparticles of construction described herein can generally have a size of 20 nm to 1020 nm. In some embodiments, composite particle size is selected from Table I.

```
TABLE I
Composite Particle Size (nm)
  5-1020
40-300
  5-505
  5-105
50-105
50-505
  50-1020
100-505
100-1020
```

Additionally, composite particles of antimicrobial compositions described herein, including composite nanoparticles, can exhibit polydipsersity (PDI) less than 0.15 or less than 0.1. In some embodiments, polydispersity of the composite particles ranges from 0.03 to 0.15 or 0.05 to 0.1

As described further herein, composite particles can be formed by several techniques. In some embodiments, composite particles are formed according to flash nano-precipitation methods described in U.S. Pat. No. 8,137,699 which is incorporated herein by reference in its entirety. Suitable flash nano-precipitation methods are also described in Johnson et al. termed “Flash NanoPrecipitation” (FNP), Johnson, B. K., et al., AIChE Journal (2003) 49:2264-2282. Flash nano-precipitation is especially effective for the production of nanoparticles from hydrophobic compounds. Where hydrophobicity is defined as having a log P greater than 3.5 or an aqueous solubility of less than 1 mg/ml. Composite particles, in some embodiments, are produced by the process layer by layer Flash NanoPrecipitation described by Pagels, R. F. et al., Journal of Controlled Release (2015), 219 pp. 519-535 and International Patent Application Number PCT/US2015/036060 (Publication Number WO 2015/200054), which is incorporated herein by reference in its entirety. In some embodiments, release rates of encapsulated actives are tuned by hydrophobic ion pairing as described by Pinkerton (Pinkerton, N. M., et al., “Formation of Stable Nanocarriers by in Situ Ion Pairing during Block-Copolymer-Directed Rapid Precipitation.” Molecular Pharmaceutics 10(1): 319-328 (2013). In some embodiments, the release rates of encapsulated actives are tuned by conjugation to produce prodrugs as described by Mayer (patent application 60/589,164 filed 19 Jul. 2004), which is incorporated herein in its entirety.

Composite particles described herein may also be produced by “Layer by Layer Flash NanoPrecipitation” or by the use of an embodiment of the “Flash NanoPrecipitation” process to encapsulate hydrophillic actives. This process allows for the encapsulation of hydrophilic components into nanoparticle form. This process has been described in full in the following patent application: PCT/US15/36060 filed Jun. 16, 2015 which is incorporated herein by reference in its entirety. This technique is especially effective for the production of nanoparticles from hydrophilic compounds, where hydrophophilicity is defined as having a log P smaller than −1 or an aqueous solubility of greater than 10 mg/ml.

Flash NanoPrecipitation and Layer by Layer Flash NanoPrecipitation are especially effective techniques because they can encapsulate actives such that the final loading of the antimicrobial agent and the adjuvant are greater than 10 wt. % of the entire nanoparticle. In some embodiments, final loading of the antimicrobial agent and the adjuvant are greater than 25 wt. % or greater than 50 wt. % of the entire nanoparticle. Final loading of the antimicrobial agent and adjuvant can also have a value selected from Table II.

TABLE II
Antimicrobial/Adjuvant Loading (wt. % of nanoparticle)
≧75
10-95
25-90
25-95
40-95
50-95
60-95

They can produce final loadings of antimicrobial agent and the adjuvant are greater than 25 wt % of the entire nanoparticle. These loadings have been difficult to achieve by alternate nanoparticle assembly processes.

In some embodiments, a method of making composite particles comprises providing a solution stream comprising amphiphilic stabilizer, antimicrobial agent and adjuvant in a process solvent and providing a non-process solvent stream. The solution stream and the non-process solvent stream are delivered to a chamber for mixing at one or more rates sufficient to flash precipitate composite particles comprising a core component encapsulated by a surface component. The core component comprises the antimicrobial agent and adjuvant, and the surface component comprises the amphiphilic stabilizer.

Composite particles described herein can be incorporated into microparticles, larger monoliths, ointments, foams sprays, catheters, hydrogels, surfaces, surgical equipment, tissue engineered products, adhesives, aerosols, medical devices. Additionally, composite particles of antimicrobial agent/antimicrobial adjuvant architecture described herein can exhibit microparticle dimensions. In such embodiments, microparticle size can be generally selected from Table III.

TABLE III
Composite Microparticle Size (μm)
1-400
2-300
4-100
4-40

Microparticles can be formulated by the aggregation of composite nanoparticles described above.

Microparticles can be formulated by emulsion followed by stripping techniques as reviewed by Pagels and Prud'homme (J. Controlled Release (2015) in press), or as described by Coombes et al. (J. Controlled Release (1993) 52, 311-320), European Patent Application 2 241 309 A2 (2010), U.S. Pat. Nos. 6,291,013 and 7,291,348 each of which is incorporated herein by reference. These techniques are applicable for the encapsulation of hydrophobic compounds. The techniques are also applicable for the encapsulation of hydrophilic compounds when used with water/oil/water emulsification processes.

Microparticles can be formed from the aggregation of pre-formed composite nanoparticles, where the nanoparticles have been formed by one of the processes described above. The aggregation is achieved by incorporating the nanoparticles into a solvent phase in which they are stable, and into which has been incorporated a binder. The solvent phase is emulsified and then the volatile solvent is removed to solidify the resulting microparticle. The microparticle is, thereby, formed as a cluster or aggregate of the smaller composite nanoparticles.

Microparticles can be also formed by spray drying nanoparticles with an appropriate binder to form an aggregate. The appropriate binder may be hydrophilic to promote the release of the aggregated nanoparticles for applications such as aerosol delivery. Such a process is described by D'Addio (D'Addio, S. M.; Chan, J. G. Y.; Kwok, P. C. L.; Benson, B. R.; Prud'homme, R. K.; Chan, H. K. Aerosol Delivery of Nanoparticles in Uniform Mannitol Carriers Formulated by Ultrasonic Spray Freeze Drying. Pharmaceutical Research 2013, 30, 2891-2901), which is not intended to be limiting. An appropriate binder may be hydrophobic to form a microparticle for depot delivery and sustained release. An appropriate binder would be polylactide-coglycolide polymers.

Alternatively, composite particles having architecture described herein can be formulated by lipid precipitation processes as described by United States Patent Application Publication 20130037977 or U.S. Pat. No. 6,500,461 each of which is incorporated herein by reference.

Composite particles described herein employ a surface component that can encapsulate the core comprising the antimicrobial agent and antimicrobial adjuvant. The surface component comprises one or more amphihilic stabilizers. An amphiphilic stabilizer is a compound having a molecular weight greater than about 500 that has a hydrophilic region and a hydrophobic region. Preferably the molecular weight is greater than about 1,000, or greater than about 1,500, or greater than about 2,000. Higher molecular weight moieties, e.g., 25,000 g/mole or 50,000 g/mole, may be used. “Hydrophobic” is defined as above. “Hydrophilic” in the context of the present invention refers to moieties that have solubility in aqueous solution of at least 1.0 mg/ml. Thus, in the amphiphilic, the hydrophobic region, if taken alone, would exhibit a solubility in aqueous medium of less than 0.05 mg/ml and the hydrophilic region, if taken alone, would exhibit a solubility in aqueous medium of more than 1 mg/ml. Amphiphilic stabilizers can be formed of copolymers. Non-limiting examples include copolymers of polyethylene glycol and polycaprolactone.

In some embodiments, the stabilizer is a copolymer of a hydrophilic block coupled with a hydrophobic block. Nanoparticles according to process described herein can be formed with graft, block or random amphiphilic copolymers. These copolymers can generally have a molecular weight between 1,000 g/mole and 50,000 g/mole or more, or between about 3,000 g/mole to about 25,000 g/mole, or at least 2,000 g/mole. Alternatively, amphiphilic copolymers used in this invention exhibit a water surface tension of at least 50 dynes/cm2 at a concentration of 0.1 wt %.

Hydrophilic block(s) and hydrophobic block(s) of amphiphilic copolymers of stabilizers described herein can generally have molecular weights provided in Table IV.

TABLE IV
Hydrophilic and Hydrophobic Molecular Weight

| Hydrophilic Block | Hydrophobic Block |
|---|---|
| 100-1,000 | 100-1,000 |
| 100-4,000 | 100-4,000 |
| 100-10,000 | 100-10,000 |
| 100-20,000 | 100-20,000 |
| >20,000 | >20,000 |
| 100-40,000 | 100-40,000 |

Depending on specific composite particle construction, molecular weight of hydrophilic and hydrophobic blocks of amphiphilic copolymer can be varied in any combination. For example, hydrophilic and hydrophobic blocks of Table III can be provided in any combination to construct amphiphilic copolymer suitable for use as composite particle stabilizer.

Examples of suitable hydrophobic blocks in an amphiphilic copolymer include but are not limited to the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate, cellulose acetate succinate, hydroxypropylmethylcellulose phthalate, poly(D,L lactide), poly (D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Ilium, L., Davids, S. S. (eds.) Polymers in Controlled Drug Delivery, Wright, Bristol, 1987; Arshady, J. Controlled Release (1991) 17:1-22; Pitt, Int. J. Phar. (1990) 59:173-196; Holland, et al., J. Controlled Release (1986) 4:155-180); hydrophobic peptide-based polymers and copolymers based on poly(L-amino acids) (Lavasanifar, A., et al., Advanced Drug Delivery Reviews (2002) 54:169-190), poly(ethylene-vinyl acetate) (“EVA”) copolymers, silicone rubber, polyethylene, polypropylene, polydienes (polybutadiene, polyisoprene and hydrogenated forms of these polymers), maleic anhydride copolymers of vinyl methylether and other vinyl ethers, polyamides (nylon 6,6), polyurethane, poly(ester urethanes), poly(ether urethanes), poly(ester-urea). Particularly preferred polymeric blocks include poly(ethylenevinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) For non-biologically related applications particularly preferred polymeric blocks include polystyrene, polyacrylates, and butadienes.

Examples of suitable hydrophilic blocks in an amphiphilic copolymer include but are not limited to the following: carboxylic acids including acrylic acid, methacrylic acid, itaconic acid, and maleic acid; polyoxyethylenes or poly ethylene oxide; polyacrylamides and copolymers thereof with dimethylaminoethylmethacrylate, diallyldimethylammonium chloride, vinylbenzylthrimethylammonium chloride, acrylic acid, methacrylic acid, 2-acrylamido-2-methylpropane sulfonic acid and styrene sulfonate, polyvinyl pyrrolidone, starches and starch derivatives, dextran and dextran derivatives; polypeptides, such as polylysines, polyarginines, polyglutamic acids; poly hyaluronic acids, alginic acids, polylactides, polyethyleneimines, polyionenes, polyacrylic acids, and polyiminocarboxylates, gelatin, and unsaturated ethylenic mono or dicarboxylic acids.

Preferably the blocks are either diblock or triblock repeats. Preferably, block copolymers for this invention include blocks of polystyrene, polyethylene, polybutyl acrylate, polybutyl methacrylate, polylactic acid, copolymers of polylactic-polyglycolic acid, polycaprolactone, polyacrylic acid, polyoxyethylene and polyacrylamide. A listing of suitable hydrophilic polymers can be found in Handbook of Water-Soluble Gums and Resins, R. Davidson, McGraw-Hill (1980).

In graft copolymers, the length of a grafted moiety can vary. Preferably, the grafted segments are alkyl chains of 12 to 32 carbons or equivalent to 6 to 16 ethylene units in length. In addition, the grafting of the polymer backbone can be useful to enhance solvation or nanoparticle stabilization properties. A grafted butyl group on the hydrophobic backbone of a diblock copolymer of a polyethylene and polyethylene glycol should increases the solubility of the polyethylene block. Suitable chemical moieties grafted to the block unit of the copolymer comprise alkyl chains containing species such as amides, imides, phenyl, carboxy, aldehyde or alcohol groups. One example of a commercially available stabilizer is the Hypermer family marketed by Uniqema Co. The amphiphilic stabilizer could also be of the gelatin family such as the gelatins derived from animal or fish collagen.

Amphiphilic stabilizers of the surface component can also be formed, in-part or whole, of acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate, cellulose acetate succinate, hydroxypropylmethylcellulose phthalate, and the polymers poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) Polymers in Controlled Drug Delivery, Wright, Bristol, 1987; Arshady, J. Controlled Release (1991) 17:1-22; Pitt, Int. J. Phar. (1990) 59:173-196; Holland, et al. J. Controlled Release (1986) 4:155-180); hydrophobic peptide-based polymers and copolymers based on poly(L-amino acids) (Lavasanifar, A., et al., Advanced Drug Delivery Reviews (2002) 54:169-190), poly(ethylene-vinyl acetate) (“EVA”) copolymers, silicone rubber, polyethylene, polypropylene, polydienes (polybutadiene, polyisoprene and hydrogenated forms of these polymers), maleic anhydride copolymers of vinyl-methylether and other vinyl ethers, polyamides (nylon 6,6), polyurethane, poly(ester urethanes), poly(ether urethanes), poly(ester-urea). Particularly preferred polymeric hydrophobes include poly(ethylenevinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) For non-biologically related applications particularly preferred polymeric species include polystyrene, polyacrylates, and butadienes acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate, cellulose acetate succinate, hydroxypropylmethylcellulose phthalate, poly(D,L lactide), poly (D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) Polymers in Controlled Drug Delivery, Wright, Bristol, 1987; Arshady, J. Controlled Release (1991) 17:1-22; Pitt, Int. J. Phar. (1990) 59:173-196; Holland, et al., J. Controlled Release (1986) 4:155-180); hydrophobic peptide-based polymers and copolymers based on poly(L-amino acids) (Lavasanifar, A., et al., Advanced Drug Delivery Reviews (2002) 54:169-190), poly(ethylene-vinyl acetate) (“EVA”) copolymers, silicone rubber, polyethylene, polypropylene, polydienes (polybutadiene, polyisoprene and hydrogenated forms of these polymers), maleic anhydride copolymers of vinyl methylether and other vinyl ethers, polyamides (nylon 6,6), polyurethane, poly(ester urethanes), poly(ether urethanes), poly(ester-urea). Particularly preferred polymeric blocks include poly(ethylenevinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) For non-biologically related applications particularly preferred polymeric blocks include polystyrene, polyacrylates, and butadienes. Composite particles can also contain amphiphilic copolymer including but are not limited to the following: carboxylic acids including acrylic acid, methacrylic acid, itaconic acid, and maleic acid; polyoxyethylenes or poly ethylene oxide; polyacrylamides and copolymers thereof with dimethylaminoethylmethacrylate, diallyldimethylammonium chloride, vinylbenzylthrimethylammonium chloride, acrylic acid, methacrylic acid, 2-acrylamido-2-methylpropane sulfonic acid and styrene sulfonate, polyvinyl pyrrolidone, starches and starch derivatives, dextran and dextran derivatives; polypeptides, such as polylysines, polyarginines, polyglutamic acids; poly hyaluronic acids, alginic acids, polylactides, polyethyleneimines, polyionenes, polyacrylic acids, and polyiminocarboxylates, gelatin, and unsaturated ethylenic mono or dicarboxylic acids.

Composite particles described herein can be modified with targeting functional moieties. Targeting functional moieties include those that influence the localization of the composite particles and the localization of corresponding associated actives during treatment. Targeting functional moieties can comprise, but are not limited to, the following species and/or derivatives and/or combinations thereof including mannose, vancomycin, polymycin B, zinc(II)-bis(dipicolylamine), sorbitol, or dipicolylamine containing or related compounds, maltose or thiomaltose or maltrodextrin containing or related compounds, viruses, virus components, antibiotics, antibodies, lectins, nucleic acids, carbohydrates, sugars, peptides, proteins, magnetically active materials, and cationic or anionic moieties. Functional moieties can target but are not limited to bacterial cell surface components, cell walls, peptidoglycan, liposaccharides, proteins, receptors, flagella, extracellular components, biofilms, cell associated nucleic acids, or localized microbial markers, such as surrounding cells or matrix components associated with infections. In some embodiments, amphiphilic stabilizer of the surface component has one or more reactive sites unto which a targeting molecule can be attached after composite particles synthesis. In such embodiments, composite nanoparticles can be surface functionalized with varying densities of targeting moieties. Composite particles can also be targeted through passive action to sites of infection, such as by changing nanocarrier diameter of surface properties. Composite particles can be targeted through other active means, such as through an external magnetic field.

It is especially advantageous that the amphiphilic polymers can be pre-functionalized with the targeting agent prior to assembly into nanoparticles. This enables better quantification of targeting ligand concentration than can be achieved by post-functionalizing pre-formed nanoparticles. Assembly of pre-functionalized amphiphilic polymers into nanoparticles can be readily accomplished by Flash NanoPrecipitation or by emulsion-stripping techniques.

Composite particle targeting, and therapy progress can be aided or monitored through the use of visualization or contrast agents.

A wide variety of imaging agents can be used for the invention. Imaging agents include radioactive markers such as for PET or SPECT imaging, fluorescent markers, dyes or photoacoustic active markers, magnetically active markers for MRI imaging, X-ray contrast agents. Agents include markers that can be used for magnetic resonance, nuclear, ultrasound, elastrographic, photoacoustic, radiographic, optical, tactile, and thermographic imaging. Agents include but are not limited to superparamagnetic iron oxide (SPIO), gadolinium, manganese, radioactive iodine, copper, zirconium, indium, yttrium, technetium, rhenium, gallium and fluorine, metals and metal oxides including but not limited to gold, palladium, iron, cobalt and ferrite.

A wide variety of antimicrobial agents can be used for the invention. Antimicrobial agents can be used to treat bacterial, fungal, viral, and parasitic infections. Antimicrobial agents can be used to treat symptoms or disease, including those caused by bacteria, archaea, fungi, protozoa, algae and/or viruses. Antimicrobial agents with molecular weight selected from Table V may be used in composite particles described herein.

TABLE V
Antimicrobial Agent Molecular Weight (Daltons)
100-10,000,000
250-10,000,000
10-1,000,000
100-200,000

In some embodiments, antimicrobial agents include those that function by killing microorganisms or by slowing microbial growth by more than 50% or more than 75%.

Antimicrobial agents include those but are not limited to those that are used to treat bacterial infections. Antimicrobials include but are not limited to aminoglycosides, ansamycins, carbacephem, carbapenems, cephalosporins, glycopeptides, lincosamides, lipopeptide, macrolides, monobactams, nitrofurans, oxazolidinones, penicillins, polypeptides, puinolones, fluoroquinolones, sulfonamides, tetracyclines, and antimycobacterials. Antimicrobials include but are not limited to amikacin, gentamicin, kanamycin, neomycin, netilmicin, tobramycin, paromomycin, streptomycin, spectinomycin, ansamycins, geldanamycin, herbimycin, rifaximin, carbacephem, loracarbef, carbapenems, ertapenem, doripenem, imipenem, cilastatin, meropenem, cephalosporins, cefadroxil, cefazolin, cefalotin or cefalothin, cephalexin, cefaclor, cefamandole, cefoxitin, cefprozil, cefuroxime, cefixime, cefdinir, cefditoren, cefoperazone, cefoperazone, cefotaxime, cefpodoxime, ceftazidime, ceftibuten, ceftizoxime, ceftriaxone, cephalosporins, cefepime, ceftaroline fosamil, ceftobiprole, teicoplanin, vancomycin, telavancin, dalbavancin, oritavancin, lincosamides, clindamycin, lincomycin, daptomycin, azithromycin, clarithromycin, dirithromycin, erythromycin, roxithromycin, troleandomycin, telithromycin, spiramycin, monobactams, aztreonam, nitrofurans, furazolidone, nitrofurantoin, oxazolidinone, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, penicillin G, temocillin, ticarcillin, bacitracin, colistin, polymyxin B, ciprofloxacin, enoxacin, gatifloxacin, gemifloxacin, levofloxacin, lomefloxacin, moxifloxacin, nalidixic acid, nisin, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilamide, sulfasalazine, sulfisoxazole, sulfonamidochrysoidine, demeclocycline, doxycycline, minocycline, oxytetracycline, tetracycline, clofazimine, dapsone, capreomycin, cycloserine, ethambutols, thionamide, isoniazid, pyrazinamide, rifampicin/rifampin, rifabutin, rifapentine, streptomycin, arsphenamine, fosfomycin, fusidic acid, metronidazole, mupirocin, platensimycin, quinupristin/dalfopristin, thiamphenicol, tigecyclines, tinidazole, and trimethoprims.

Antimicrobials include those but are not limited to those to treat fungal infections. Antimicrobials include but are not limited to polyenes, imidazole, triazole, and thiazole antifungals, allylamines, and echinocandins. Antimicrobials include but are not limited to amphotericin B, candicidin, filipin, natamycin, nystatin, rimocidin, azole antifungals, canesten, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaeonazole, fluconazole, isavuconazole, itraconazole, posaconazole, ravuconazole, terconazole, voriconazole, abafungin, allylamines, amorolfin, butenafine, naftifine, terbinafine, echinocandins, anidulafungin, caspofungin, micafungin, echinocandins, benzoic acid, ciclopirox, flucytosine, griseofulvin, haloprogin, tolnaftate, undecylenic acid, and crystal violet.

Antimicrobials include those but are not limited to those to treat viral infections. Antimicrobials include but are not limited to abacavir, ziagen, trizivir, kivexa/epzicom, acyclovir, acyclovir, adefovir, amantadine, amprenavir, ampligen, arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, fusion inhibitors, ganciclovir, ibacitabine, imunovir, idoxuridine, imiquimod, indinavir, inosine, integrase inhibitor, interferon type III, interferon type II, interferon type I, interferon, iamivudine, lopinavir, loviride, maraviroc, moroxydine, methisazone, nelfinavir, nevirapine, nexavir, nucleoside analogues, novir, oseltamivir, peginterferon alfa-2a, penciclovir, peramivir, pleconaril, podophyllotoxin, raltegravir, reverse transcriptase inhibitor, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, and HIV drugs.

Antimicrobials include those but are not limited to those to treat parasitic infections. Antimicrobials include but are not limited to antinematodes, anticestodes, antitrematodes, antiamoebics, and antiprotozoals. Antimicrobials include but are not limited to mebendazole, pyrantel pamoate, thiabendazole, diethylcarbamazine, ivermectin, niclosamide, praziquantel, albendazole, praziquantel, rifampin, amphotericin B, melarsoprol, eflornithine, metronidazole, tinidazole, miltefosine, and antimalarial drugs.

Antimicrobials include those but are not limited to those that treat infections using antivirulence pathways.

In addition to being part of the core component, antimicrobial agents may also be attached to the surface component of the composite particles. Antimicrobial agent may be covalently linked, physically associated, ionically associated, or surface absorbed on the surface of composite particles.

A wide range of antimicrobial adjuvants can be used for the invention. Antimicrobial adjuvant with molecular weight selected from Table VI may be used in composite particles described herein.

TABLE VI
Antimicrobial Adjuvant Molecular Weight (Daltons)
100-10,000,000
250-10,000,000
10-1,000,000
100-200,000

As described herein, antimicrobial adjuvants can enhance the efficacy of the antimicrobial agent. In some embodiments, for example, the adjuvant can comprise one or more agents for permeabilizing or otherwise penetrating the antimicrobial cell wall. In other embodiments, the adjuvant can be an agent that improves or enhances transport of the antimicrobial agent across the cell wall and/or limits degradation of the antimicrobial additive. An adjuvant may also serve to limit efflux of antimicrobial additive(s) out of the microbial cell. In some embodiments, the adjuvant can be cell wall targeting agent.

The antimicrobial adjuvant can be an agent that when co-encapsulated with an antimicrobial agent results in a composite particle having an antimicrobial combination index less than 1 or less than 0.5 when compared to particles encapsulated with only antimicrobial agent and particles encapsulated with only antimicrobial adjuvant. In some embodiments, the antimicrobial adjuvant provides a composite particle having an antimicrobial index less than 1 or less than 0.5 when compared to unencapsulated antimicrobial additive(s) and unencapsulated antimicrobial adjuvant(s). In further embodiments, the antimicrobial combination index of composite particles described herein can be selected from Table VII.

TABLE VII
Combination Index of Composite Particles

<0.25
<0.1
<0.01
<0.001

As described further herein, the values in Table VII can be relative to particles encapsulated with only antimicrobial agent and particles encapsulated with only antimicrobial adjuvant.

Generally, adjuvants of composite particles described herein do not exhibit antimicrobial activity. In some embodiments, for example, adjuvant(s) of composite particles do not exhibit antimicrobial activity at one or more of the adjuvant concentrations provided in Table VIII.

TABLE VIII
Adjuvant Concentration

| | |
|---|---|
| 1000 | mg/ml |
| 100 | mg/ml |
| 10 | mg/ml |
| 0.1 | mg/ml |
| 0.01 | mg/ml |
| 1 | μg/ml |
| 0.1 | μg/ml |
| 1 | ng/ml |
| 0.1 | ng/ml |
| 0.01 | ng/ml |
| 1 | pg |

Antimicrobial adjuvants include, but are not limited to, quorum-sensing active or communication signaling actives. Quorum-sensing actives can agonize or antagonize population coordinated behavior of microbes. Quorum-sensing actives can alter the behavior of microbes from modulating cell-cell signaling communication. Quorum-sensing actives can change microbial biological states. Communication signal actives can activate or inhibit pathways that can be used in cell communication to alter the behavior or metabolic state microbes, sensitizing microbes to antimicrobial treatments. Antimicrobial adjuvants include but are not limited to acyl-homoserine lactone, diffusible signal factor, autoinducer peptide, oligopeptide, fatty acid, butyrolactone, furanones, thiolactones, epinephrine, norepinephrine, small molecule, peptide, protein, and metabolite compounds and related compounds. Antimicrobial adjuvants include but are not limited to autoinducer-1, autoinducer-2, cholerae autoinducer-1, 6-gingerol, garlic, garlic extract, furanones C-30 and C-56, baicalin hydrate, cinnamaldehyde, hamamelitannin, mBTL, mCTL, cis-2-decenoic acid, (Z)-4-bromo-5-(bromomethylene)-3-methylfuran-2(5H)-one, diffusible signaling factors T8-DSF, T10-DSF, T11-DSF, T12-DSF, T13-DSF, T14-DSF, C8-DSF, C10-DSF, C11-DSF, C12-DSF, DSF, C13-DSF, C14-DSF, C15-DSF, and S12-DSF, 3-oxo-C6-HSL, panax gingseng, panax gingseng extract, PQS analogs, BHL analogs, OdDHL analogs, phenylacetyl homoserine lactones, phenoxyacetyl homoserine lactones, phenylpropionyl homoserine lactones, 3-oxo-c12-(2-aminophenol), penicillic acid, 4-nitro-pyridine-n-oxide, TP-1, TP-5, patulin, salicylic acid, tiaprofenic acid, donepezil, chlorzoxazone, nifuroxazide, indoramin, iron, bile salts, 2-heptyl-4-hydroxyquinolines, anacardic acids, 3-acyltetramic acids, Ea-CAI-1 analogs, farnesol, AIP-1, AIP-2, AIP-3, CSP, ComX, GBAP, PQS, 3′-sialyl lactose, p-nitro-o-Cl-Ph mannoside, salicylideneaniline, virustatin, hamamelitannin, baicalein, 2-AIT, ureidothiopehene, C6HSL, C14-HSL, C4HSL, C8HSL, C16-C20HSL, ajoene, ayurveda spice clove, brominated thiophenone, caffeine, curcumin, honaucin A, iberin, limonoids, isolimonic acid, ichangin, N,N′-disubstituted imidazolium salts, naringenin, propolis, demethoxy encecalin, kojic acid, microlins, hymenialdisin, pyrogallol, protoanemonin, sesquiterpene lactones, D-amino acids, and compounds derived from or related from the above compounds.

Antimicrobial adjuvants, in some embodiments, include enzyme inhibiting compound(s), various sugars, pro-drug(s) and/or metal colloids, such as silver containing colloids. Antimicrobial adjuvants may also be employed to alter or control antimicrobial agent release rate.

Antimicrobial adjuvants include but are not limited to those that target the CqsA, CqsS, HapR, LuxU, LuxO, LuxS, LuxI, LuxP, LuxR, LsrBm LsRB, LsrK, QsecC, AI-1, AI-2, AI-3, Qrr1-4, AphA, LuxN TCP, CTX, VPS proteins, pathways, and related proteins and pathways.

Antimicrobial adjuvants include but are not limited to those that target LasR, LasI, QscR, RhlR, RhlI, RelA, RpoS, PQS, MvaT, VfR, GacA, RsmA, RsaL, elastase, exotoxin A, alkaline protease, Xcp secretion, LasA, elastase, pyocyanin rhamnolipid, lectin, proteins, pathways, and related proteins and pathways.

Antimicrobial adjuvants include but are not limited to those that target AgrA, AgrB, AgrC, AgrD, ComP, ComQ, ComA, proteins, pathways, and related proteins and pathways.

Antimicrobial adjuvants include but are not limited to those that target Cph1, Efg1, Tec1, HSGs, Nrg1, Tup1, Rfg1, Tup1, Rbf1, CyR1, Tpk2, Nrg1, Sok1, Cup9, Ubr1, MAP kinase, Ras/cAMP-dependent, and Rim101 proteins, NAD, NADH, NADPH pathways, and related proteins and pathways.

Antimicrobial adjuvants include but are not limited to those that target V. cholerae, P. aeruginosa, S. aureus, or C. albicans signaling pathways.

Antimicrobial adjuvants include but are not limited to those that degrade compounds, signaling molecules, or biofilms. Antimicrobial adjuvants include but are not limited to lactonase, acylase, AHL-oxidase, acylase, oxidoreductase, AHL-lactonase, proteases, DNase, proteinase K, alginate lyase, dispersin B, lysozyme, collagenase, trypsin, chymotrypsin, nuclease, chitinase, and EPS-degrading proteins and related proteins.

In addition to being part of the core component, antimicrobial adjuvants may also be attached to the surface component of the composite particles. Antimicrobial adjuvant may be covalently linked, physically associated, ionically associated, or surface absorbed on the surface of composite particles.

Antimicrobial adjuvants include but are not limited to nitric oxide and nitric oxide donating prodrugs. Nitric oxide molecules can transform bacteria into a more susceptible metabolic state, by causing oxidative stress, or by enhancing the response of the innate immune system towards the microbial infection. Nitric oxide releasing compounds include but are not limited to diazeniumdiolates, nitrosohydroxylamines, C-bound diazeniumdiolates, N-bound diazeniumdiolates, N-niazeniumdiolated intermediates, unsubstituted diazeniumdiolates, O-subsituted diazeniumdiolates, S-nitrosothiols, nitrosamines, NO-metal complexes, organic nitries, and organic nitrates compounds and related compounds. Antimicrobial adjuvants include but are not limited to carbon monoxide and carbon monoxide donating prodrugs.

Antimicrobial adjuvants include but are not limited to nutrients, sugars, peptides, proteins, carbohydrates, fats, metals, ions, phosphates, salts. Antimicrobial adjuvants include but are not limited to gluclose, mannitol, fructose, glycerol, pyruvate, gluconate, ribose, arabinose, glycolate, galactarate. Antimicrobial adjuvants include, but are not limited to, fosfomycin, tellurite, pivmecillinam, echinomycin, clavulanic acid, sulbactam, tazobactam, cilastatin, bacitracin, vancomycin, cycloserine, colistin, polymyxin B, polymyxin B2, eugenol, phenylpropanoids, exopolysaccharides, D-amino acids and derivatives thereof.

Antimicrobial adjuvants include but are not limited to metals or metallic ions. Metals or metallic ions include but are not limited to silver, copper, iron, lead, zinc, bismuth, gold, and aluminium ions, salts, or colloids.

Antimicrobial adjuvants include but are not limited to biofilm inhibitors and dispersants. Antimicrobial adjuvants include but are not limited to AHLs, AIP and agr inducers, DSF, AI-2, D-amino acids, nitric oxide, nutrients, carbon and nitrogen limitation causing compounds, carbon limiting compounds, oxygen limiting compounds, iron, EPS-degrading enzymes, chitinase, nuclease, and dispersin. Antimicrobial adjuvants include but are not limited to molecules that target quorum-sensing, c-di-GMP, Bd1A, chemotaxis and aerotaxis genes, HNOX, LapG, LapA, YhjH, MxdB, ArcA, Crp, RdbA, pqs systems, exopolysaccharide lyase, ChiA, ChiB, DNA, and N-acetylglucoasmine.

Antimicrobial adjuvants include but are not limited to molecules that inhibit ATB-binding cassettes, major facilitator superfamily, resistance-nodulation-division, small multidrug resistance, multidrug and toxic compound extrusion, outer membrane, periplasm, cytoplasmic membrane, membrane fusion protein, proteins and related proteins. Antimicrobial adjuvants include but are not limited to drugs that target Cml, CmlA, CmlB, Cmlv, Cmx, Cmr, CmA, MdFa, OqXAB, RND, MexEF-OprN, CmeABC, AcrAB-TolC, Flo, FloR, pp-Flo, FexA, Mef(A), MacAB-TolC, MtrCDE, Cme, MdfA, CmeABC, MexCD-OprJ, Msr(D), AcrAB-TolC, Mex, MdeA, RND pumps, Msr(A), Msr(C), Vga(A/B), Lsa, LmrB, Lsa(B), Tet(A), Tet(B), Tet(C), Tet(D), Tet(E), Tet(G), Tet(H), Tet(J), Tet(Y), Tet(Z), Tet(30), Tet(39), Tet(K), Tet(L), Tet38, Tet(V), Rv1258/tap, P55/Rv1410, Rv2333c, DrrAB, MdfA, MexAB-OprM, AdeABC, CmeABC, MepA, AbeM, LmrA, EmrE, NorA, NorB, MepA, PmrA, EmeA, LmrA, Lde, EfrAB, Bmr, Blt, Bmr3, CdeA, MD1, MD2, LfrA, EfpA, Rv1634, PstB, DrrAB, Mmr proteins and related proteins. Antimicrobial adjuvants include carbonylcyaninde m-chlorophenylhydrazone, valinomycin and dinitrphenol, reserpine, verapamil, omeprazole, 11 pyrrolo[1,2-a] quinaxoline derivates, tricyclic neuroleptics, phenothiazines, promethazine, flavoligan 5′-methoxy-hydnocarpin, peptidomimetics, MC-207 110, phenylalanine arginyl beta-napthylamide, alkyl/akenyl/alynl amides, PAbeta-N, quinolone, phenicol, cycline, quinazolinones, arylpiperidines, arylpiperazines, substituted polyamines, N-benzylated polyazaalkanes, N0benzylated polyaminoalkanes, iron chelators, nocardamine, GG918, biricodar, timcodar, VX-710, and cyclosporine.

Antimicrobial adjuvants include but are not limited to molecules that inhibit enzymes degradation or modification of antimicrobial agents. Antimicrobial adjuvants include but are not limited to molecules that inhibit beta-lactamases, macrolide esterases, epoxidases, acyltransferases, phosphotransferases, thioltransferases, nucleotidyltransferases, ADP-ribosyltransferases, glycosyltransferases, redox enzymes, or redox enzymes. Antimicrobial adjuvants include but are not limited to tazobactam, relebactam, cilastatin, and biphenyl tetrazoles.

As described herein, simultaneous encapsulation of antimicrobial and antimicrobial adjuvants into composite particles can be performed using a wide range of methods. Agents with the sufficient hydrophilic and hydrophobic agents can be encapsulated with the above described formation methods. Agent compositions which have agents that do not have hydrophilic and hydrophobic properties that allow for simultaneous co-encapsulation and controlled release from the composite particles can be engineered. Agents can be chemically linked to a functional group with varying hydrophobic or hydrophillic properties that will produce a final agent prodrug with the properties that permit simultaneous co-encapsulation and controlled release of each component at the necessary release rates. Agents can also be modified through ion pairing.

A “linker” refers to any covalent bond, to a divalent residue of a molecule, or to a chelator (in the case where the active is a metal ion or organic metallic compound, e.g., cisplatin) that allows the hydrophobic moiety to be attached to the active agent. The linker may be selectively cleavable upon exposure to a predefined stimulus, thus releasing the active agent from the hydrophobic moiety. The site of cleavage, in the case of the divalent residue of a molecule may be at a site within the residue, or may occur at either of the bonds that couple the divalent residue to the agent or to the hydrophobic moiety. The predefined stimuli include, for example, pH changes, enzymatic degradation, chemical modification or light exposure. Convenient conjugates are often based on hydrolyzable or enzymatically cleavable bonds such as esters, carbonates, carbamates, disulfides and hydrazones.

In some instances, the conditions under which the active performs its function are not such that the linker is cleaved, but the active is able to perform this function while still attached to the particle. In this case, the linker is described as “non-cleavable,” although virtually any linker could be cleaved under some conditions; therefore, “non-cleavable” refers to those linkers that do not necessarily need to release the active from the particle as the active performs its function.

The linker component, as described above, may be or may include a cleavable bond.

The linker may be, for example, cleaved by hydrolysis, reduction reactions, oxidative reactions, pH shifts, photolysis, or combinations thereof; or by an enzyme reaction. Some linkers can be cleaved by an intracellular or extracellular enzyme, or an enzyme resulting from a microbial infection, a skin surface enzyme, or an enzyme secreted by a cell, by an enzyme secreted by a cancer cell, by an enzyme located on the surface of a cancer cell, by an enzyme secreted by a cell associated with a chronic inflammatory disease, by an enzyme secreted by a cell associated with rheumatoid arthritis, by an enzyme secreted by a cell associated with osteoarthritis, or by a membrane-bound enzyme. In some cases, the linker can be cleaved by an enzyme that is available in a target region. These types of linkers are often useful in that the particular enzyme or class of enzymes may be present in increased concentrations at a target region. The target tissue generally varies based on the type of disease or disorder present in the subject.

The linker may also comprise a bond that is cleavable under oxidative or reducing conditions, or may be sensitive to acids. Acid cleavable linkers can be found in U.S. Pat. Nos. 4,569,789 and 4,631,190; and Blattner, et al., Biochemistry (1984) 24:1517-1524. Such linkers are cleaved by natural acidic conditions, or alternatively, acid conditions can be induced at a target site as explained in U.S. Pat. No. 4,171,563.

Examples of linking reagents which contain cleavable disulfide bonds (reducible bonds) include 1,4-di-[3′-(2′-pyridyldithio)propionamido]butane; N-succinimidyl(4-azidophenyl) 1,3′-dithiopropionate; sulfosuccinimidyl (4-azidophenyldithio)propionate; dithiobis(succinimidylpropionate); 3,3′-dithiobis(sulfosuccinimidylpropionate); dimethyl 3,3′-dithiobispropionimidate-2HCl (available from Pierce Chemicals, Rockford, Ill.).

Examples of oxidation sensitive linking reagents include, without limitation, disuccinimidyl tartarate; and disuccinimidyl tartarate (available from Pierce Chemicals).

The linker may also comprise a small molecule such as a peptide linker. Frequently, in such embodiments, the peptide linker is cleavable by base, under reducing conditions, or by a specific enzyme. The linker may be cleaved by an indigenous enzyme, or by an non-indigenous enzyme administered after or in addition to the presently contemplated compositions. A small peptide linker is pH sensitive, for example, the linker may comprise linkers selected from the group consisting of poly L-glycine; poly L-glutamine; and poly L-lysine linkers.

For example, the linker may comprise a hydrophobic polymer and a dipeptide, L-alanyl-L-valine (Ala-Val), cleavable by the enzyme thermolysin. This linker is advantageous because thermolysin-like enzyme has been reported to be expressed at the site of many tumors. A linker may also be used that contains a recognition site for the protease furin. Goyal, et al., Biochem. J. (2000) 2:247-254.

The chemical and peptide linkers can be bonded between the ligand and the agent by techniques known in the art for conjugate synthesis, i.e., using genetic engineering or chemically.

Photocleavable linkers include, for example, 1-2-(nitrophenyl)-ethyl. A photocleavable linker often permits the activation and action of the active agent in a very specific area, for example at a particular part of the target tissue. Activation (light) energy can be localized through a variety of means including catheterization, via natural or surgical openings or via blood vessels.

The linkers and techniques for providing coupling of the active to the hydrophobic moiety are similar to those that have been used previously to prepare conjugates to make actives more soluble, in contrast to their application in the present invention. In general, in the constructs of the invention, the active is often, but not always, made less soluble in aqueous solution by virtue of forming the conjugate. For example, the techniques reviewed by Greenwald, et al., for attaching PEG to small organic molecules can be adapted to the present invention. Some of these techniques are described in Greenwald, R. B., Journal of Controlled Release (2001) 74:159-171; Greenwald, R. B., et al., Journal of Medicinal Chemistry (1996) 39:424-431; and Greenwald, R. B., et al., Advanced Drug Delivery Reviews (2002) 55:217-250. In particular, paclitaxel esters have been prepared via conjugation of PEG acids to the paclitaxel molecule. These esters were demonstrated to be an especially effective linking group, as hydrolysis of the ester carbonyl bond and the subsequent release of the attached drug were shown to occur in a predictable fashion in vitro. (Greenwald, R. B., et al., Critical Reviews in Therapeutic Drug Carrier Systems (2000) 17:101-161.) The linker chemistry as applied in the present invention does not enhance solubility, but adapts the active agent for inclusion in the particulate vehicles of the invention.

The covalent attachment of proteins, vaccines or peptides to PEG can also be adapted to form the present conjugate. Such techniques are reviewed in Katre, N. V., Advanced Drug Delivery Reviews (1993) 10:91-114; Roberts, M. J., et al., Journal of Pharmaceutical Sciences (1998) 87:1440-1445; Garman, A. J., et al., Febs Letters (1987) 223:361-365; and Daly, S. M., et al., Langmuir (2005) 21:1328-1337. Coupling reactions between amino groups of proteins and mPEG equipped with an electrophilic functional group have been used in most cases for preparation of PEG-protein conjugates. The most commonly used mPEG-based electrophiles, referred to as ‘activated PEGs’ are based on reactive aryl chlorides, acylating agents and alkylating groups as described by Zalipsky, S., Advanced Drug Delivery Reviews (1995) 16:157-182; and Zalipsky, S., Bioconjugate Chem. (1995) 6:150-165. Tailoring the number of ethylene groups in the linker can additionally be used to adjust the hydrolysis rates of drug-linked ester bonds, to values appropriate for once-a-week administration. For example, Schoenmakers, et al., demonstrated the conjugation of a model paclitaxel molecule to PEG using a hydrolysable linker based on reaction between a thiol and an acrylamide. By changing the length of the linker, the time of drug release was varied between 4 and 14 days. (Schoenmakers, R. G., et al., Journal of Controlled Release (2004) 95:291-300.) Additionally, Frerot, et al., prepared a series of carbamoyl esters of maleate and succinate and studied the rate constants for neighboring group assisted alkaline ester hydrolysis. The rates of hydrolysis were found to depend on the structure of the neighboring nucleophile that attacks the ester function. (de Saint Laumer, J. Y., et al., Helvetica Chimica Acta (2003) 86:2871-2899.) By taking account of the influence of structural parameters on the rates of ester hydrolysis, hydrolysis rates may be varied over several orders of magnitude and precursors yielding the desired release profile may be designed.

In addition to ester linkages, enzymatically cleavable bonds can be used to conjugate active agents to the hydrophobic moiety. An enzymatically cleavable linker generally will comprise amino acids, sugars, nucleic acids, or other compounds which have one or more chemical bonds that can be broken via enzymatic degradation. In a recent study, a variety of amino acid spacers were employed for the conjugation of PEG to camptothecin, an anti-tumor drug. Rates of amino acid linker hydrolysis were determined to vary according to the type of amino acid spacer utilized. (Conover, C. D., et al., Anti-Cancer Drug Design (1999) 14:499-506).

Photocleavable linkers have also been extensively employed for the synthesis of conjugates for release of actives. As an example, keto-esters have been used as delivery systems for the controlled release of perfumery aldehydes and ketones. Alkyl or aryl keto esters of primary or secondary alcohols decompose upon radiation at 350-370 nm, releasing the active aldehyde. (Rochat, S., et al., Helvetica Chimica Acta (2000) 83:1645-1671.) This mechanism has been shown to successfully sustain release of the active agent. For drug delivery purposes, light energy can be localized through a variety of means including catheterization, via natural and surgical openings or via blood vessels.

As noted above, when the linker is the residue of a divalent organic molecule, the cleavage “of the linker” may be either within the residue itself, or it may be at one of the bonds that couples the linker to the remainder of the conjugate-i.e., either to the active or the hydrophobic moiety.

In some embodiments, it is unnecessary for the linker to be cleavable. In particular, if the active is functional while still coupled to the linker, there is no need to release the active from the particulate moiety. One such example would be instances wherein the active is printer's ink, which can remain in particulate form when employed.

In instances where the linker need not be cleavable, alternative organic moieties may be used to create the divalent residue, or a covalent bond directly coupling the active to the hydrophobic moiety may not be subject to cleavage under conditions contemplated in use. (By “non-cleavable” is meant that the linker will not release the active under the conditions wherein the function of the active is being performed.) Examples of non-cleavable linkers comprise, but are not limited to, (sulfosuccinimidyl 6-[alpha-methyl-alpha-(2-pyridylthio)toluamido]hexanoate; Azidobenzoyl hydrazide; N-Hydroxysuccinimidyl-4-azidosalicyclic acid; Sulfosuccinimidyl 2-(p-azidosalicylamido)ethyl-1,3-dithiopropionate; N-{4-(p-azidosalicylamido) buthy}maxima-3-(2-pyidyldithio)propionamide; Bis-[beta-(4-azidosalicylamido)ethyl]disulfide; N-hydroxysuccinimidyl-4 azidobenzoate; p-Azidophenyl glyoxal monohydrate; N-Succiminidyl-6(4-azido-2-mitrophenyl-amino)hexanoate; Sulfosuccinimidyl 6-(4-azido-2-nitrophenylamino)hexanoate; N-5-Azido-2-nitrobenzyoyloxysuccinimide; Sulfosuccinimidyl-2-(m-azido-o-mitrobenzamido)-ethyl-1,3-dithiopropionate; p-nitrophenyl-2-diazo-3,3,3-trifluoropropionate; Succinimidyl 4-(N-maleimidomethyl)cyclohexane-1-carboxylate; Sulfosuccinimidyl 4-(N-maleimidomethyl)cyclohexane-1-carboxylate; m-Maleimidobenzoyl-N-hydroxysuccinimide ester; m-Maleimidobenzoyl-N-hydroxysulfosuccinimide ester; N-Succinimidyl(4-iodoacetyl)aminobenzoate; N-Sulfosuccinimidyl(4-iodoacetyl)aminobenzoate; Succinimidyl 4-(p-malenimidophenyl)butyrate; Sulfosuccinimidyl 4-(p-malenimidophenyl)butyrate; Disuccinimidyl suberate; bis(sulfosuccinimidyl) suberate; Bis maleimidohexane; 1,5-difluoro-2,4-dinitrobenzene; dimethyl adipimidate 2HCl; Dimethyl pimelimidate-2HCl; dimethyl suberimidate-2-HCl; “SPDP”-N-succinimidyl-3-(2-pyridylthio)propionate; Sulfosuccinimidyl 4-(p-azidophenyl)butyrate; Sulfosuccinimidyl 4-(p-azidophenylbutyrate); 1-9p-azidosalicylamido)-4-(iodoacetamido)butane; 4-(p-Azidosalicylamido)butylamine (available from Pierce Chemicals).

In cases where the antimicrobial agent and/or antimicrobial adjuvant is not sufficiently hydrophobic to form uniform composite particles according to the methods outlined above (g—Flash NanoPrecipitation) and (h—Emulsion Stripping), the antimicrobial agent/adjuvant can be formulated with a counter ionic species to form a hydrophobic salt in situ that will render the active-counter ion complex amenable to forming nanoparticles by Flash NanoPrecipitation or emulsion stripping. Non-limiting examples of counter ionic species are: (±)-camphor-10-sulfonic acid, pamoic acid, cinnamic acid, palmitic acid, oleic acid, and N,N′-dibenzyl-ethylenediamine.

In some cases the release rates of drugs can be tuned by changing the properties of the composite particle core component. Properties of the core component, such as pH, local water content, local charge, tortuosity, porosity, crystallinity, and drug diffusivity can be tuned by co-encapsulating additional agents in addition to the actives. The above mentioned properties can be tuned to change the release of antimicrobial agents and antimicrobial adjuvants, as well as change the cleavage of prodrug linkages for tuning active release rates. Materials that can be co-encapsulated to tune nanoparticle properties include but are not limited any of the above listed materials.

Pharmaceutical or veterinary compositions comprising delivery vehicles of the invention are prepared according to standard techniques and may comprise water, buffered water, 0.9% saline, 0.3% glycine, 5% dextrose, iso-osmotic sucrose solutions and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like.

A two-active agent combination may be further used as a single pharmaceutical unit to determine synergistic or additive interactions with a third agent. In addition, a three-agent combination may be used as a unit to determine non-antagonistic interactions with a fourth agent, and so on.

The optimal combination ratio may be further used as a single pharmaceutical unit to determine synergistic or additive interactions with a third agent. In addition, a three-agent combination may be used as a unit to determine non-antagonistic interactions with a fourth agent, and so on.

As set forth above, the in vitro studies on cell cultures will be conducted with “relevant” cells. The choice of cells will depend on the intended therapeutic use of the agent. Only one relevant cell line or cell culture type need exhibit the required non-antagonistic effect in order to provide a basis for the compositions to come within the scope of the invention.

The therapeutic agents in the present composite particles may be formulated separately in individual compositions wherein each therapeutic agent is stably associated with appropriate delivery vehicles. These compositions can be administered separately to subjects as long as the pharmacokinetics of the delivery vehicles are coordinated so that the ratio of therapeutic agents administered is maintained at the target for treatment. Thus, it is useful to construct kits which include, in separate containers, a first composition comprising delivery vehicles stably associated with at least a first therapeutic agent and, in a second container, a second composition comprising delivery vehicles stably associated with at least one second therapeutic agent. The containers can then be packaged into the kit.

The kit will also include instructions as to the mode of administration of the compositions to a subject, at least including a description of the ratio of amounts of each composition to be administered. Alternatively, or in addition, the kit is constructed so that the amounts of compositions in each container is pre-measured so that the contents of one container in combination with the contents of the other represent the correct ratio. Alternatively, or in addition, the containers may be marked with a measuring scale permitting dispensation of appropriate amounts according to the scales visible. The containers may themselves be useable in administration; for example, the kit might contain the appropriate amounts of each composition in separate syringes. Formulations which comprise the pre-formulated correct ratio of therapeutic agents may also be packaged in this way so that the formulation is administered directly from a syringe prepackaged in the kit.

These and other embodiments are further illustrated by the following non-limiting examples.

### EXAMPLE 1

### Composite Nanoparticles

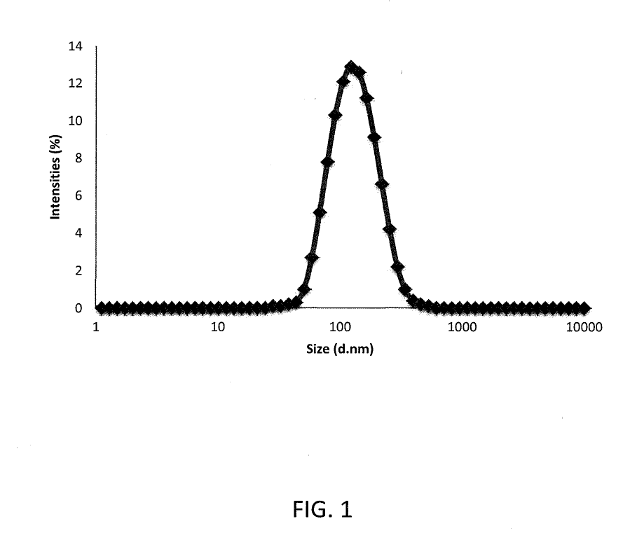

Composite nanoparticles comprising the co-encapsulation of cis-2-decenioc acid and intraconazole were prepared using Flash NanoPrecipitation by by dissolving polymeric 5 kDa polycaprolactone-block 5 kDa polyethylene glycol stabilizer, alpha-tocopherol, cis-2-decenoic acid, and intraconazole in tetrahydrofuran, and rapidly impinging dissolved components with water in a confined impingement jet. FIG. 1 illustrates the dynamic light scattering particle size distribution of the composite nanoparticles comprising cis-2-decenioc acid and intraconazole.

### EXAMPLE 2

### Composite Nanoparticles

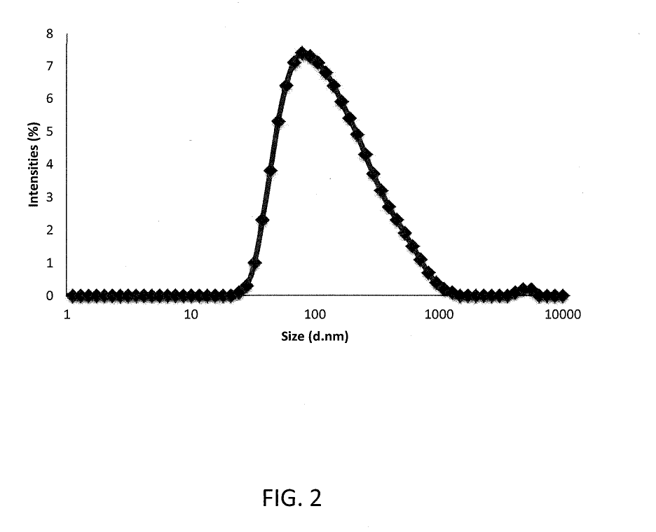

Composite nanoparticles comprising co-encapsulation of farnesol with intraconazole were prepared using Flash NanoPrecipitation, by dissolving polymeric 5 kDa polycaprolactone-block 5 kDa polyethylene glycol stabilizer, alpha-tocopherol, farnesol, and intraconazole in tetrahydrofuran, and rapidly impinging dissolved components with water in a confined impingement jet mixer. FIG. 2 illustrates the dynamic light scattering particle size distribution of the composite nanoparticles comprising farnesol and intraconazole.

### EXAMPLE 3

### Composite Nanoparticles

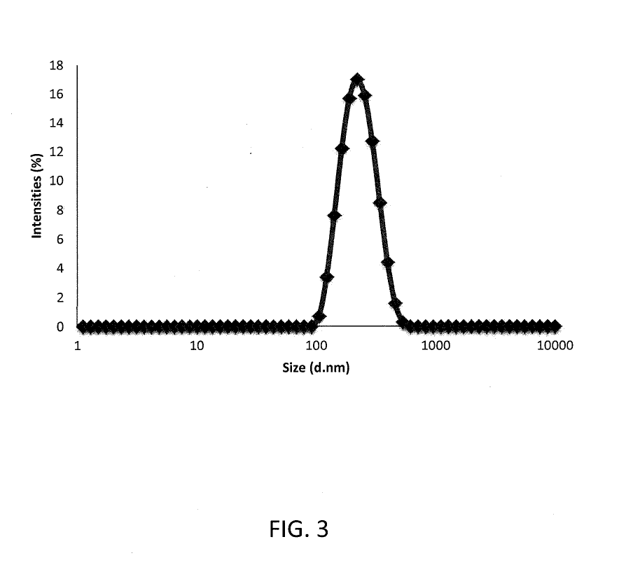

Composite nanoparticles comprising co-encapsulation of silver colloids with intraconazole were prepared using Flash NanoPrecipitation, by dissolving polymeric 5 kDa polycaprolactone-block 5 kDa polyethylene glycol stabilizer, alpha-tocopherol, oleic acid coated silver colloids, and intraconazole in tetrahydrofuran, and rapidly impinging dissolved components with water in a confined impingement jet mixer. FIG. 3 illustrates the dynamic light scattering particle size distribution of the composite nanoparticles comprising silver colloid and intraconazole.

### EXAMPLE 4

### Composite Nanoparticles

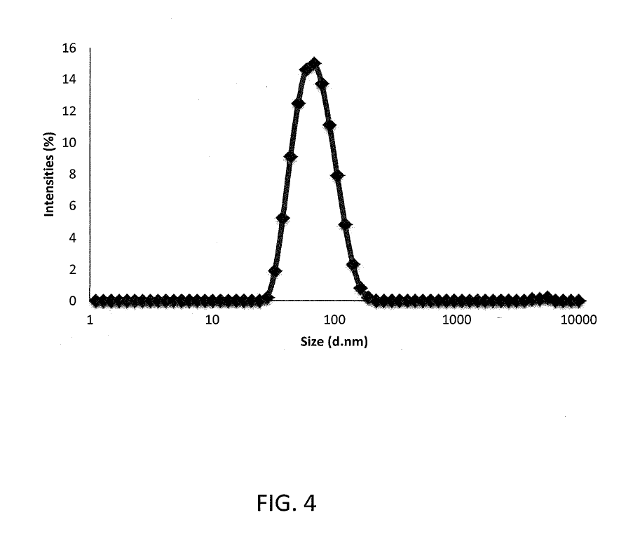

Composite nanoparticles comprising co-encapsulation of cis-2-decenoic acid with totarol were prepared using Flash NanoPrecipitation, by dissolving polymeric 5 kDa polycaprolactone-block 5 kDa polyethylene glycol stabilizer, alpha-tocopherol, cis-2-decenoic acid, and totarol in tetrahydrofuran, and rapidly impinging dissolved components with water in a confined impingement jet mixer. FIG. 4 illustrates the dynamic light scattering particle size distribution of the composite nanoparticles comprising cis-2-decenoic acid and totarol.

### EXAMPLE 5

### Composite Nanoparticles

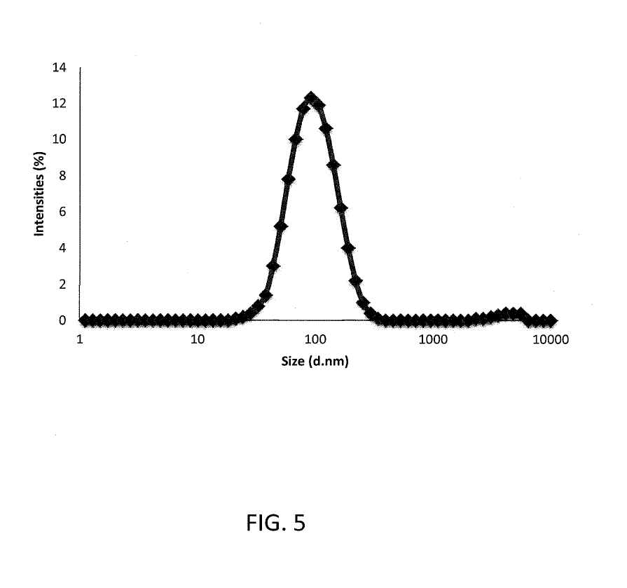

Composite nanoparticles comprising co-encapsulation of polymyxin with totarol were prepared using Flash NanoPrecipitation, by dissolving polymeric 5 kDa polycaprolactone-block 5 kDa polyethylene glycol stabilizer, alpha-tocopherol, polymyxin ion paired with lauryl sulfate, and totarol in tetrahydrofuran, and rapidly impinging dissolved components with water in a confined impingement jet mixer. FIG. 5 illustrates the dynamic light scattering particle size distribution of the composite nanoparticles comprising polymyxin and totarol.

### EXAMPLE 6

### Composite Nanoparticles

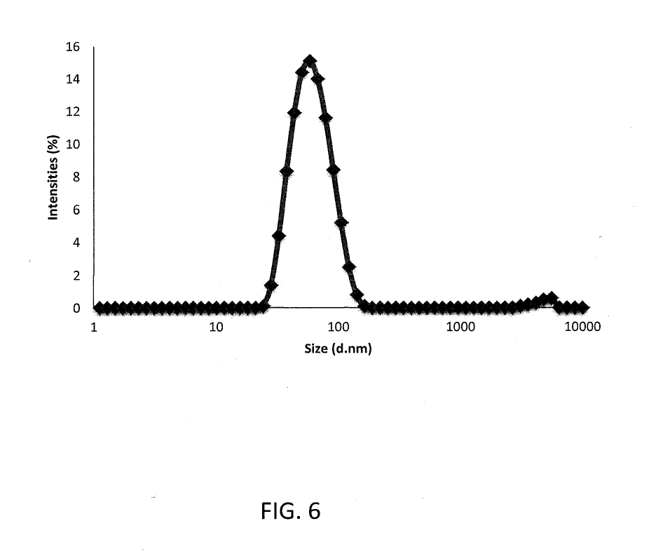

Composite nanoparticles comprising co-encapsulation of cis-2-decenoic acid with rifampicin prodrugs were prepared using Flash NanoPrecipitation, by dissolving polymeric 5 kDa polycaprolactone-block 5 kDa polyethylene glycol stabilizer, alpha-tocopherol, cis-2-decenoic acid, and rifampicin conjugated with Vitamin E succinate by the cleavable ester bond, in tetrahydrofuran, and rapidly impinging dissolved components with water in a confined impingement jet mixer. FIG. 6 illustrates the dynamic light scattering particle size distribution of the composite nanoparticles comprising cis-2-decenoic acid and rifampicin prodrugs.

### EXAMPLE 7

### Composite Nanoparticles

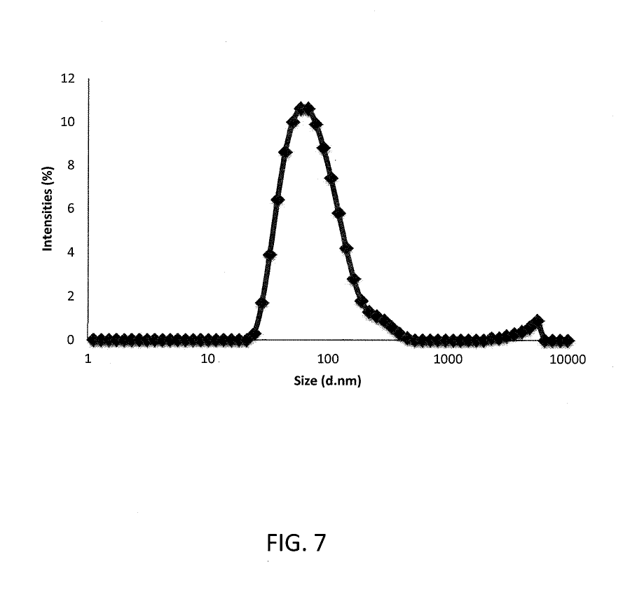

Composite nanoparticles comprising co-encapsulation of silver colloids with rifampicin prodrugs were prepared using Flash NanoPrecipitation, by dissolving polymeric 5 kDa polycaprolactone-block 5 kDa polyethylene glycol stabilizer, alpha-tocopherol, oleic acid coated silver colloids, and rifampicin conjugated with Vitamin E succinate by the cleavable ester bond, in tetrahydrofuran, and rapidly impinging dissolved components with water in a confined impingement jet mixer. FIG. 7 illustrates the dynamic light scattering particle size distribution of the composite nanoparticles comprising silver colloids and rifampicin prodrugs.

### EXAMPLE 8

### Composite Nanoparticles

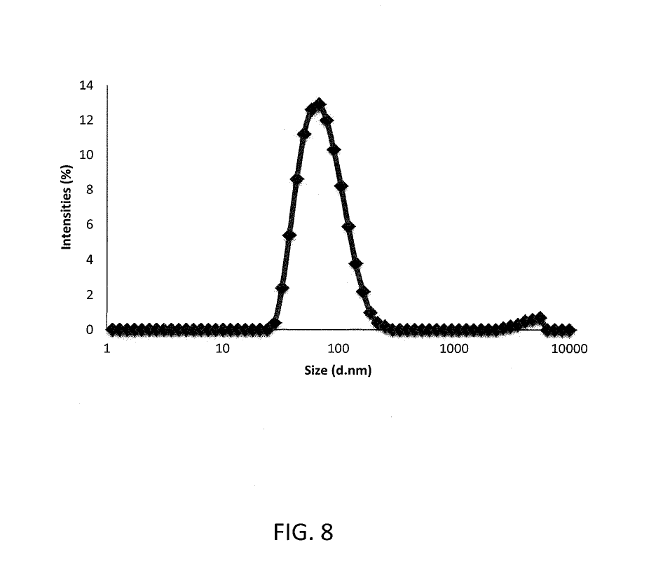

Composite nanoparticles comprising co-encapsulation of polymyxin with rifampicin prodrugs were prepared using Flash NanoPrecipitation, by dissolving polymeric 5 kDa polycaprolactone-block 5 kDa polyethylene glycol stabilizer, alpha-tocopherol, polymyxin ion-paired with lauryl sulfate, and rifampicin conjugated with Vitamin E succinate by the cleavable ester bond, in tetrahydrofuran, and rapidly impinging dissolved components with water in a confined impingement jet mixer. FIG. 8 illustrates the dynamic light scattering particle size distribution of the composite nanoparticles comprising polymyxin and rifampicin prodrugs.

### EXAMPLE 9

### Composite Nanoparticles

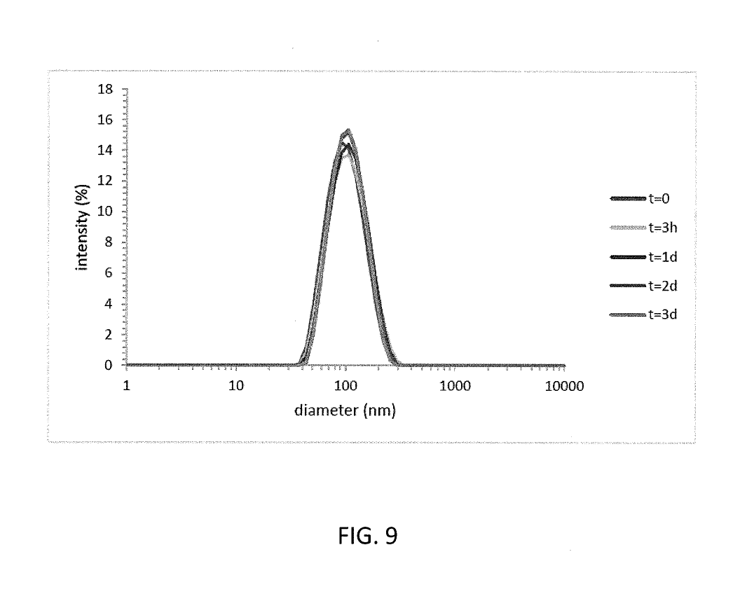

Composite nanoparticles comprising co-encapsulation of silver colloids with totarol were prepared using Flash NanoPrecipitation, by dissolving polymeric 3.9 kDa polycaprolactone-block 5.5 kDa polyethylene glycol stabilizer, alpha-tocopherol, oleic acid coated silver colloids, and totarol, and rapidly impinging dissolved components with water in a confined impingement jet mixer. The composite nanoparticles were subsequently stored in water at room temperature for a time period of three days. FIG. 9 illustrates dynamic light scattering particle size distribution of the composite nanoparticles taken at time zero, three hours, 1 day, 2 day and 3 days of the storage. As illustrated in FIG. 9, the composite nanoparticles were stable and did not agglomerate.

### EXAMPLE 10

### Composite Nanoparticles

Composite nanoparticles co-encapsulating oleic acid coated silver colloids, totarol and various co-core compositions were prepared by Flash NanoPrecipitation by dissolving polymeric 3.9 kDa polycaprolactone-block 5.5 kDa polyethylene glycol stabilizer, a “co-core”, oleic acid coated silver colloids and totarol, and rapidly impinging dissolved components with water. The stabilizer is defined as “PCL-PEG” in Table IX. The co-core is defined as “co-core” in the chart. HC1100 stands for Capa HC1100 grade polycaprolactone in the chart. 2 k PCL stands for 2 kDa polycaprolactone polymer in the chart. OA stands for oleic acid in the chart. VES stands for vitamin E succinate in the chart. Silver stands for oleic acid coated silver colloids in the chart. Z-diameter is the intensity-weighted diameter of nanoparticles in nanometers. PDI is polydispersity index.

TABLE IX

| | Stabilizer | | Core | | Core | | Core | | NP Properties | |
|---|---|---|---|---|---|---|---|---|---|---|
| Form. | Copolymer | (mg/mL) | Co-core | (mg/mL) | Adjuvant | (mg/mL) | Antibiotic | (mg/mL) | Z-diameter (nm) | PDI |
| 3 | PCL-PEG | 5 | HC1100 | 2.5 | silver | 2.5 | totarol | 2.5 | 134.7 | 0.092 |
| 4 | PCL-PEG | 5 | HC1100 | 5 | silver | 2.5 | totarol | 2.5 | 152 | 0.054 |
| 5 | PCL-PEG | 5 | 2K PCL | 2.5 | silver | 2.5 | totarol | 2.5 | 122 | 0.098 |
| 6 | PCL-PEG | 5 | 2K PCL | 5 | silver | 2.5 | totarol | 2.5 | 159.6 | 0.093 |
| 5 | PCL-PEG | 5 | OA | 2.5 | silver | 2.5 | totarol | 2.5 | 121 | 0.067 |
| 6 | PCL-PEG | 5 | OA | 5 | silver | 2.5 | totarol | 2.5 | 180.9 | 0.036 |
| 5 | PCL-PEG | 5 | VES | 2.5 | silver | 2.5 | totarol | 2.5 | 148.5 | 0.067 |
| 6 | PCL-PEG | 5 | VES | 5 | silver | 2.5 | totarol | 2.5 | 191.5 | 0.051 |

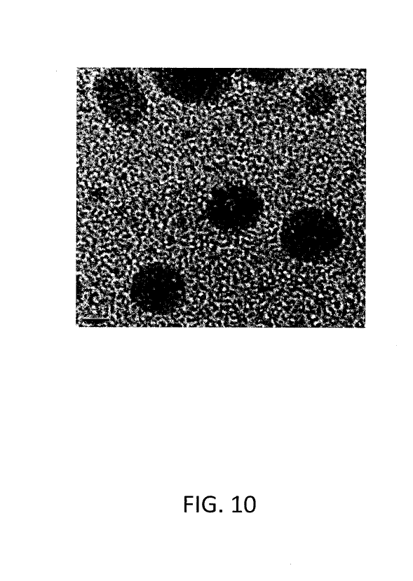

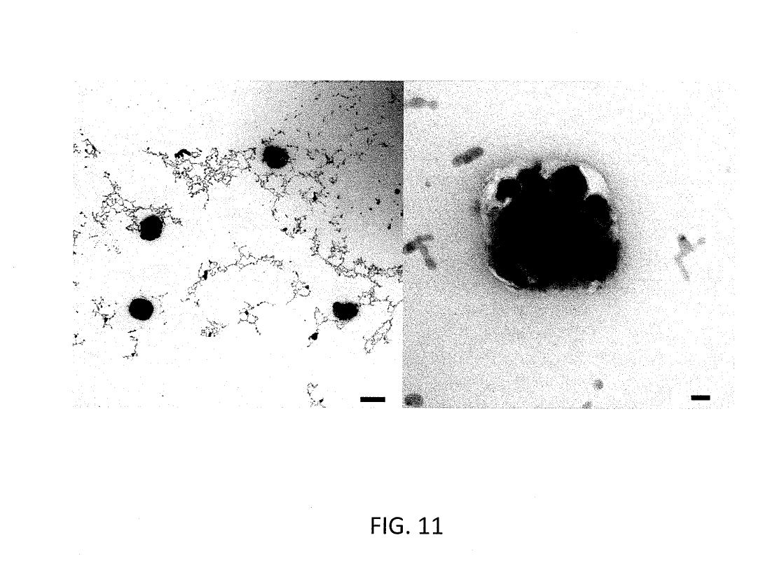

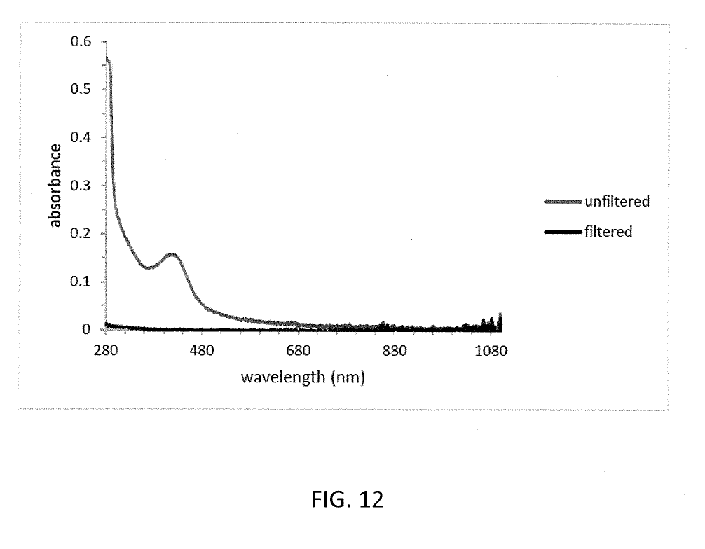

FIG. 10 is a transmission electron microscopy (TEM) image of oleic acid coated silver colloids employed in the present example. The scale bar is 5 nm. Moreover, FIG. 11 provides TEM images of the composite nanoparticles co-encapsulating oleic acid coated silver colloids and totarol. Additionally, FIG. 12 illustrates absorbance spectra of composite nanoparticles that co-encapsulate oleic acid coated silver colloids with totarol and the absorbance spectra of the flow-through fraction of nanoparticles that co-encapsulate oleic acid coated silver colloids with totarol when passed through a 10 kDa ultrafiltration filter. When filtered, only unencapsulated components are in the flow-through fraction. The flow-through fraction does not contain totarol and does not contain silver. Both totarol and silver are co-encapsulated in the same particle population.

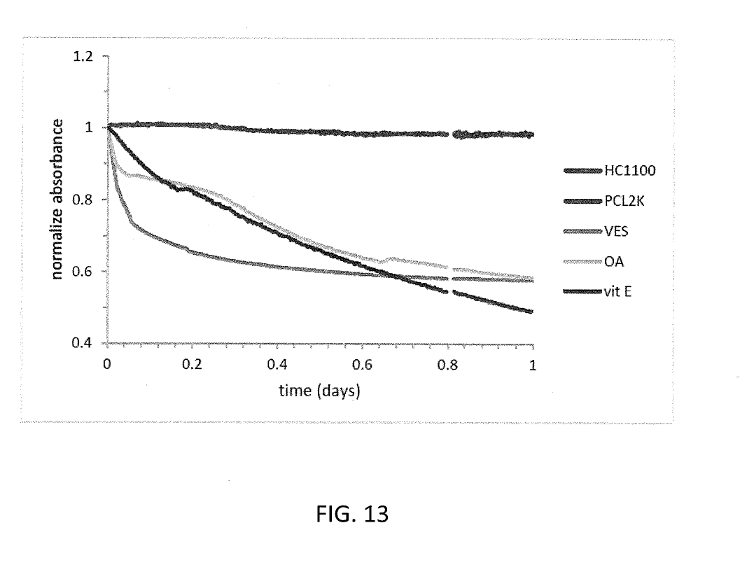

FIG. 13 illustrates the normalized absorbance peak maxima in the visible wavelength region over time when the composite nanoparticles of this example are diluted into phosphate buffered saline. The decrease in absorbance is due to the release of drugs into the buffered saline. The use of different type of “co-core” and the used nanoparticles can tine the relative rates of drug release over time.

Table X characterizes the biological activity of the composite nanoparticles made with various Flash NanoPrecipitation organic feed stream compositions.

TABLE X

| | PCL-PEG | Vitamin E | silver | totarol | MIC |
|---|---|---|---|---|---|
| Formulation | (mg/mL) | (mg/mL) | (mg/mL) | (mg/ml) | (μg/mL) |
| 1 | 5 | 2.5 | 2.5 | 2.5 | 125 |
| 2 | 5 | 2.5 | 2.5 | 0 | >500 |
| 3 | 5 | 2.5 | 0 | 2.5 | >500 |
| 4 | 5 | 0 | 2.5 | 2.5 | 15.6 |
| 5 | 5 | 0 | 2.5 | 0 | 500 |
| 6 | 5 | 0 | 0 | 2.5 | 125 |

The MIC stands for the minimal inhibitory concentration of the constructs against Staphylococcus aureus Newman in a broth microdilution assay. The reported MIC is based on the concentration of therapuetic actives, or the concentration of antimicrobial active plus concentration of antimicrobial adjuvant used in the cell assay. The MIC was determined with cells grown in lysogeny broth media. Silver stands for oleic acid coated silver colloids in the chart. PCL-PEG stands for 3.9 kDa polycaprolactone-block 5.5 kDa polyethylene glycol. Vitamin E stands for alpha-tocopherol. Formulations 2 and 3, or particles encapsulating “Vitamin E and totarol” or only “Vitamin E and silver”, have MIC greater than 500 μg/mL. Formulation 1, or particles that co-encapsulate altogether “Vitamin E and totarol and silver” have MIC of 125 μg/mL. The particles in Formulation 1 have a combination index of at most 0.25, showing synergy upon co-encapsulation of both totarol and silver. Formulation 5, or particles encapsulating “silver” have a MIC of 500 μg/mL. Formulation 6, or particles encapsulating “totarol” have a MIC of 125 μg/mL. Formulation 4, or particles encapsulating “silver and totarol” have a MIC of 15.6 μg/mL. The particles in Formulation 4 have a combination index of 0.078, showing synergy upon co-encapsulation of both totarol and silver.

The following equation is used to determine the combination index (CI).

$$CI = \frac{[ A ]}{[ {A}_{0} ]} + \frac{[ Adj ]}{[ {Adj}_{0} ]}$$

[A] stands for concentration the of the antimicrobial active in the composite nanoparticle that co-encapsulates antimicrobial active and antimicrobial adjuvant at the composite nanoparticle's MIC. [Adj] stands for the concentration of the antimicrobial adjuvant in the composite nanoparticle that co-encapsulates antimicrobial active and antimicrobial adjuvant at the composite nanoparticle's MIC. [A₀] stands for the concentration of the antimicrobial active in nanoparticles encapsulating only the antimicrobial active, at this nanoparticle's MIC. [Adj₀] stands for the concentration of the antimicrobial adjuvant in nanoparticles encapsulating only the antimicrobial adjuvant, at this nanoparticle's MIC. CI below one indicates synergy. Variations of the CI equation include but are not limited [A₀] that stands for the MIC concentration of the unencapsulated antimicrobial agent and [Adj₀] that stands for the MIC concentration of the unencapsulated antimicrobial adjuvant.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. An antimicrobial composition comprising:
a plurality of composite nanoparticles, the composite nanoparticles comprising a core component and a surface component, the core component including at least one antimicrobial agent and at least one antimicrobial adjuvant and the surface component comprising one or more amphiphilic stabilizers.

2. The antimicrobial composition of claim 1, wherein the surface component encapsulates the core component including the antimicrobial agent and antimicrobial adjuvant.

3. The antimicrobial composition of claim 1, wherein the one or more amphiphilic stabilizers comprise amphiphilic copolymer.

4. The antimicrobial composition of claim 3, wherein a targeting molecule is attached to the amphiphilic copolymer.

5. The antimicrobial composition of claim 4, wherein the targeting molecule is selected from the group consisting of zinc(II)-bis(dipicolylamine), polymyxin, vancomycin, maltose, sorbitol and derivatives thereof.

6. The antimicrobial composition of claim 1, wherein the composite nanoparticles have a size of 40 nm to 300 nm.

7. The antimicrobial composition of claim 1, wherein the composite nanoparticles exhibit polydispersity of 0.3 to 0.15.

8. The antimicrobial composition of claim 3, wherein the amphiphilic copolymer comprises a hydrophobic block and hydrophilic block, the hydrophobic and hydrophilic blocks each having a molecular weight of 100 to 40,000.

9. The antimicrobial composition of claim 1, wherein the composite nanoparticles exhibit a combination index (CI) of less than 1.

10. The antimicrobial composition of claim 1, wherein the composite nanoparticles exhibit a CI of less than 0.25.

11. The antimicrobial composition of claim 1, wherein loading of the antimicrobial agent and antimicrobial adjuvant is 25 to 95 weight percent of a composite nanoparticle.

12. The antimicrobial composition of claim 1, wherein loading of the antimicrobial agent and antimicrobial adjuvant is 40 to 95 weight percent of a composite nanoparticle.

13. The antimicrobial composition of claim 1, wherein loading of the antimicrobial agent and antimicrobial adjuvant is 50 to 95 weight percent of a composite nanoparticle.

14. The antimicrobial composition of claim 1, wherein the antimicrobial adjuvant does not exhibit antimicrobial activity at a concentration of 0.1 mg/ml.

15. The antimicrobial composition of claim 1, wherein the antimicrobial adjuvant is a quorum sensing compound.

16. The antimicrobial composition of claim 1, wherein the antimicrobial adjuvant comprises polymyxin or polymyxin derivative.

17. The antimicrobial composition of claim 1, wherein the antimicrobial adjuvant is selected from the group consisting of a biofilm dispersing compound, a nitric oxide releasing compound, a sugar, an enzyme inhibiting compound and a cell wall permeabilizing agent.

18. The antimicrobial composition of claim 1, wherein the antimicrobial adjuvant comprises a metal colloid.

19. The antimicrobial composition of claim 18, wherein the metal colloid comprises silver.

20. The antimicrobial composition of claim 1, wherein the antimicrobial agent, antimicrobial adjuvant or both are conjugated as a pro-drug to tune hydrophobicity of the antimicrobial agent or antimicrobial adjuvant.

21. The antimicrobial composition of claim 1, wherein the antimicrobial agent, antimicrobial adjuvant or both are conjugated as a pro-drug to control release rate from the composite nanoparticles.

22. The antimicrobial composition of claim 1, wherein the antimicrobial agent, antimicrobial adjuvant or both are ion-paired to tune hydrophobicity of the antimicrobial agent or antimicrobial adjuvant.

23. The antimicrobial composition of claim 1, wherein the antimicrobial agent, antimicrobial adjuvant or both are ion paired to control release rate from the composite nanoparticles.

24. The antimicrobial composition of claim 1, wherein the antimicrobial agent is active against gram negative bacteria.

25. The antimicrobial composition of claim 1, wherein the antimicrobial agent is active against gram positive bacteria.

26. The antimicrobial composition of claim 1, wherein the antimicrobial agent is active against fungi or protozoa.

27. The antimicrobial composition of claim 1, wherein the composite nanoparticles aggregate into microparticles having a size of 1 μm to 400 μm.
